(19) United States
(12) Patent Application Publication
(10) Pub. No.: US 2025/0025526 A1
(43) Pub. Date: Jan. 23, 2025

(54) COMBINATION THERAPY FOR THE TREATMENT OF FRIEDREICH'S ATAXIA

(71) Applicant: Stealth BioTherapeutics Inc., Needham, MA (US)

(72) Inventors: David Brown, Needham, MA (US); Alyssa Handler, Needham, MA (US); Laura Kropp, Needham, MA (US); Henry Hess, Needham, MA (US)

(73) Assignee: Stealth BioTherapeutics Inc., Needham, MA (US)

(21) Appl. No.: 18/899,153

(22) Filed: Sep. 27, 2024

Related U.S. Application Data
(63) Continuation of application No. PCT/US2024/034769, filed on Jun. 20, 2024.
(60) Provisional application No. 63/522,551, filed on Jun. 22, 2023.

(51) Int. Cl. A61K 38/07; A61K 31/277; A61P 25/28

(52) U.S. Cl. CPC A61K 38/07; A61K 31/277; A61P 25/28

(57) ABSTRACT

The present disclosure provides a combination therapy for treating, preventing, inhibiting, ameliorating, or delaying the onset of Friedreich's ataxia in a subject in need thereof comprising administering to the subject both elamipretide and omaveloxolone or pharmaceutically acceptable salts, stereoisomers, mixtures of stereoisomers, tautomers, hydrates, and/or solvates thereof. The present disclosure further provides an embodiment of a combination product suitable for administration to said subject.

### CROSS-REFERENCE TO RELATED APPLICATIONS

This application claims the benefit of and priority to U.S. Provisional Application No. 63/522,551, filed on Jun. 22, 2023, the entire contents of which are incorporated herein by reference for any and all purposes.

### TECHNICAL FIELD

The present application relates generally to a combination therapy and related product for treating, preventing, inhibiting, ameliorating and/or delaying the onset of Friedreich's ataxia, in a subject in need thereof and/or reducing the severity of such disease or its associated disorders and/or conditions in a subject. In particular, the present application is related to combining the administration of both elamipretide and omaveloxolone to a subject in need thereof suffering from, or believed to be suffering from, Friedreich's ataxia.

### INTRODUCTION

The following description is provided to assist the understanding of the reader. None of the information provided or references cited herein is admitted as being prior art to the compounds, compositions, products and/or methods disclosed herein.

Friedreich's ataxia (FA) is a fatal, monogenic, autosomal recessive disease caused by mutations in the gene encoding the nuclear encoded mitochondrial protein frataxin. Tissues in both the peripheral and central nervous systems are affected in FA, and include the dentate nucleus, Clark's column, spinocerebellar tract and dorsal root ganglia. Progressive degeneration of these tissues leads to a worsening ataxia, which for most patients ends in loss of independent ambulation by the third decade of life.

Loss of frataxin function results in the disruption of iron-sulfur cluster biosynthesis, mitochondrial iron overload, oxidative stress, impaired aerobic electron transport chain respiration and cell death in the brain, spinal cord, dorsal root ganglia, and heart.

Ferroptosis is an iron-dependent type of cell death that is biochemically distinct from apoptosis and typically accompanied by a large amount of iron accumulation and lipid peroxidation during the cell death process. Ferroptosis-inducing factors can directly or indirectly affect glutathione peroxidase through different pathways, resulting in a decrease in antioxidant capacity and accumulation of lipid reactive oxygen species (ROS) in cells, ultimately leading to oxidative cell death. Recent studies have shown that ferroptosis is closely related to the pathophysiological processes of many diseases, such as tumors, nervous system diseases, ischemia-reperfusion injury, kidney injury, and blood diseases. Decreased expression of frataxin (FXN) is associated with mitochondrial dysfunction, mitochondrial iron accumulation, and increased oxidative stress. Recent studies have shown that frataxin, which modulates iron homeostasis and mitochondrial function, is a key regulator of ferroptosis. As such, ferroptosis has been identified as a therapeutic target for treating Friedreich's ataxia.

Mitochondrial iron overload leads to impaired intra-mitochondrial metabolism and a defective mitochondrial respiratory chain. A defective mitochondrial respiratory chain leads to increased free radical generation and oxidative damage, which may be considered as mechanisms that compromise cell viability. Some evidence suggests that frataxin might detoxify ROS via activation of glutathione peroxidase and elevation of thiols. (See e.g., Calabrese et al., Journal of the Neurological Sciences, 233 (1): 145-162 (June 2005)).

Symptoms of Friedreich's ataxia typically begin between the ages of 5 and 15 years, although they sometimes appear in adulthood. The first symptom to appear is usually gait ataxia, or difficulty walking. The ataxia gradually worsens and slowly spreads to the arms and the trunk. There is often loss of sensation in the extremities, which may spread to other parts of the body. Other features include loss of tendon reflexes, especially in the knees and ankles. Most people with Friedreich's ataxia develop scoliosis, which often requires surgical intervention for treatment. Dysarthria (slowness and slurring of speech) develops and can get progressively worse. Many individuals with later stages of Friedreich's ataxia develop hearing and vision loss.

Heart disease often accompanies Friedreich's ataxia, such as hypertrophic cardiomyopathy, myocardial fibrosis (formation of fiber-like material in the muscles of the heart), and cardiac (heart) failure. Heart rhythm abnormalities such as tachycardia (fast heart rate) and heart block (impaired conduction of cardiac impulses within the heart) are also common. Other symptoms that may occur include chest pain, shortness of breath, and heart palpitations.

Many patients with Friedreich's ataxia will exhibit a slow decline in visual acuity in later stages of the disease. The most common ophthalmic manifestation of Friedreich's ataxia is optic neuropathy. In some cases, severe/catastrophic visual loss is experienced.

The rate of progression of the disease varies from person to person. Generally, within 10 to 20 years after the appearance of the first symptoms, the person is confined to a wheelchair, and in later stages of the disease individuals may become completely incapacitated. Friedreich's ataxia can shorten life expectancy, and heart disease is the most common cause of death.

There is no known cure for Friedreich's ataxia. Generally, therapies involve treatment of the symptoms. Because patients with Friedreich's ataxia are at risk of developing heart disease, they are often prescribed medications such as beta blockers, ACE inhibitors and/or diuretics. Because it is believed that damage caused by oxidative stress is involved in the progression of Friedreich's ataxia, antioxidants such as vitamin E, idebenone and coenzyme Q10 are often co-administered to persons diagnosed or suspected of having Friedrich's ataxia. These compounds have been used in various clinical trials.

Omaveloxolone is a second generation synthetic oleanane triterpenoid that is believed to exhibit antioxidative and anti-inflammatory activity. Omaveloxolone has the structure:

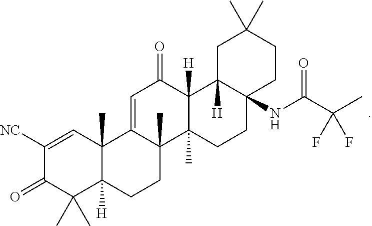

Omaveloxolone is a potent activator of the essential transcription factor, NF-E2-related factor 2 (Nrf2), and is believed to target redox-sensitive cysteine residues on the regulatory molecule Keap1 and thereby rescues Nrf2 from degradation. Omaveloxolone is currently in clinical development for treatment of a variety of indications, and recently was approved by the United States Food and Drug Administration (FDA) for the treatment of Friedreich's ataxia.

Elamipretide is a tetrapeptide with the amino acid sequence: H-D-Arg-2′,6′Dmt-Lys-Phe-NH₂, where 2′,6′-Dmt is the amino acid 2′,6′-dimethyltyrosine. Elamipretide has the structure:

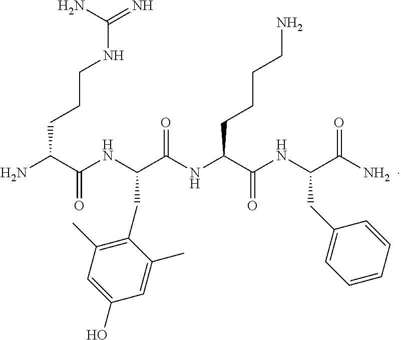 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(N)=O]

Elamipretide is also referred to in the scientific literature as SS-31, bendavia and MTP-131. Elamipretide is a cell-permeable tetrapeptide that transiently localizes to the inner mitochondrial membrane, where it reversibly binds to cardiolipin to improve membrane stability, enhance ATP synthesis in several organs, and reduce reactive oxygen species (ROS) production. Elamipretide is being investigated in the clinic for the treatment of a variety of diseases/disorders, including Friedreich's ataxia (See: WO2015/017861 U.S. Pat. No. 10,835,573; and the website for clinicaltrials.gov).

Notwithstanding the foregoing, there is no cure for Friedreich's ataxia and there remains a need for better treatments and therapies to address this patient population.

### SUMMARY

In one aspect, the present disclosure provides a method for treating, preventing, inhibiting, ameliorating or delaying the onset of Friedreich's ataxia or the signs or symptoms of reduced frataxin levels or activity in a subject in need thereof, comprising administering to the subject a therapeutically effective amount of both elamipretide and omaveloxolone, or pharmaceutically acceptable salts, stereoisomers, mixtures of stereoisomers, tautomers, hydrates, and/or solvates thereof.

In some embodiments, the subject displays reduced levels of frataxin expression compared to a normal control subject.

In some embodiments, the Friedreich's ataxia comprises one or more of muscle weakness, loss of coordination, vision impairment, hearing impairment, slurred speech, curvature of the spine, diabetes, and heart disorders.

In some embodiments, administration of elamipretide and omaveloxolone to a subject diagnosed as having, or suspected of having, Friedreich's ataxia is effective to increase intracellular adenosine triphosphate (ATP) levels in tissue in a subject.

In some embodiments, administration of elamipretide and omaveloxolone to a subject diagnosed as having, or suspected of having, Friedreich's ataxia is effective to increase or maintain frataxin levels in the subject.

In some embodiments, each of the elamipretide and omaveloxolone is independently administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly.

In some embodiments, the elamipretide and omaveloxolone are administered simultaneously, or substantially simultaneously. In some embodiments, the elamipretide and omaveloxolone are administered sequentially in either order. In some embodiments, the elamipretide and omaveloxolone are administered separately.

In some embodiments, the elamipretide and omaveloxolone are administered by different routes of administration. In some embodiments, the elamipretide is administered subcutaneously. In some embodiments, the omaveloxolone is administered orally.

In some embodiments, the subject has been diagnosed as having Friedreich's ataxia. In some embodiments, the subject is human.

In some embodiments, the administration of both elamipretide and omaveloxolone produces a synergistic therapeutic effect.

In one aspect, the present disclosure provides a kit comprising both elamipretide and omaveloxolone, or pharmaceutically acceptable salts, stereoisomers, mixtures of stereoisomers, tautomers, hydrates, and/or solvates thereof, each in a form suitable for administration to a subject diagnosed as having, or suspected of having, Friedreich's ataxia.

In one aspect, the present disclosure provides a composition, formulation or medicament comprising both elamipretide and omaveloxolone, or pharmaceutically acceptable salts, stereoisomers, mixtures of stereoisomers, tautomers, hydrates, and/or solvates thereof, in a form suitable for administration to a subject diagnosed as having, or suspected of having, Friedreich's ataxia.

### BRIEF DESCRIPTION OF THE DRAWINGS

FIG. 1 is a graphical illustration of data obtained comparing healthy patient cells treated with 1 μM RSL3 to induce ferroptosis followed by treatment with elamipretide alone, omaveloxolone alone or the combination of elamipretide and omaveloxolone.

FIG. 2 is a graphical illustration of data obtained comparing Friedreich's ataxia (FA) patient fibroblasts treated with 500 nM RSL3 to induce ferroptosis followed by treatment with elamipretide alone, omaveloxolone alone or the combination of elamipretide and omaveloxolone.

### DETAILED DESCRIPTION

It is to be appreciated that certain aspects, modes, embodiments, variations and features of the present disclosure are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, N Y, 1987); and Meth. Enzymol., Vols. 154 and 155, Wu & Grossman, and Wu, Eds., respectively.

### I. Definitions

Definitions of specific functional groups and chemical terms are described in more detail below. The chemical elements are identified in accordance with the Periodic Table of the Elements, GAS version, Handbook of Chemistry and Physics, 7Sh Ed., inside cover. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

It is to be appreciated that certain aspects, modes, embodiments, variations and features of the technology are described below in various levels of detail in order to provide a substantial understanding of the present disclosure. The definitions of certain terms as used in this specification are provided below. Unless defined otherwise, all technical and scientific terms used herein generally have the same meaning as commonly understood by one of ordinary skill in the art to which this technology belongs.

As used in this specification and the appended embodiments, the singular forms “a”. “an” and “the” include plural references unless the content clearly dictates otherwise. For example, reference to “a cell” includes a combination of two or more cells, and the like.

As used herein, “administering” or the “administration” of an agent (i.e. a therapeutic agent) or compound/drug product (including a composition (i.e. a formulation or medicament)) to a subject includes any route of introducing or delivering to a subject a compound/drug product to perform its intended function. Administration may be carried out by any suitable route, such as oral administration. Administration can be carried out subcutaneously. Administration can be carried out intravenously. Administration can be carried out intraocularly. Administration can be carried out systemically. Alternatively, administration may be carried out topically, intranasally, intraperitoneally, intradermally, ophthalmically, intrathecally, intracerebroventricularly, iontophoretically, transmucosally, intravitreally, or intramuscularly. Administration includes self-administration, the administration by another or administration by use of a device (e.g., an infusion pump).

As used herein, to “ameliorate” or “ameliorating” a disease, disorder or condition refers to results that, in a statistical sample or specific subject, make the occurrence of the disease, disorder or condition (or a sign, symptom or condition thereof) better or more tolerable in a sample or subject administered a therapeutic agent relative to a control sample or subject.

As used herein, the term “amino acid” includes both a naturally occurring amino acid and a non-natural amino acid. The term “amino acid,” unless otherwise indicated, includes both isolated amino acid molecules (i.e., molecules that include both, an amino-attached hydrogen and a carbonyl carbon-attached hydroxyl) and residues of amino acids (i.e., molecules in which either one or both an amino-attached hydrogen or a carbonyl carbon-attached hydroxyl are removed). The amino group can be alpha-amino group, beta-amino group, etc. For example, the term “amino acid alanine” can refer either to an isolated alanine H-Ala-OH or to any one of the alanine residues H-Ala-,-Ala-OH, or -Ala-. Unless otherwise indicated, all amino acids found in the compounds described herein can be either in D or L configuration. An amino acid that is in D configuration may be written such that “D” precedes the amino acid abbreviation. For example, “D-Arg” represents arginine in the D configuration. According to convention, if there is no “D” or “L” that precedes the amino acid, the amino acid is assumed to be of the “L” configuration. The term “amino acid” includes salts thereof, including pharmaceutically acceptable salts. Any amino acid can be protected or unprotected. Protecting groups can be attached to an amino group (for example alpha-amino group), the backbone carboxyl group, or any functionality of the side chain. As an example, phenylalanine protected by a benzyloxycarbonyl group (Z) on the alpha-amino group would be represented as Z-Phe-OH. Amino acid protecting groups are well known in the art. A comprehensive review of amino acid protecting groups can be found in: Isidro-Llobet et al., Chem. Rev. (2009) 109:2455-2504.

With the exception of the N-terminal amino acid, all abbreviations of amino acids (for example, Phe) in this disclosure stand for the structure of —NH—C(R)(R′)—CO—, wherein R and R′ each is, independently, hydrogen or the side chain of an amino acid (e.g., R=benzyl and R′=H for Phe). Accordingly, phenylalanine is H-Phe-OH. The designation “OH” for these amino acids, or for peptides (e.g., Lys-Val-Leu-OH) indicates that the C-terminus is the free acid. The designation “NH₂” in, for example, H-Phe-D-Arg-Phe-Lys-NH₂ indicates that the C-terminus of the protected peptide fragment is amidated. In each case, an “H” preceding an amino acid or peptide indicates that the amine of the amino acid or peptide N-terminus is unmodified (i.e. is —NH₂). Further, certain R and R′, separately, or in combination as a ring structure, can include functional groups that may require protection during the liquid phase or solid phase synthesis.

Where the amino acid has isomeric forms, it is the L form of the amino acid that is represented unless otherwise explicitly indicated as D form, for example, D-Arg. Notably, many amino acid residues are commercially available in both D- and L-form. For example, D-Arg is a commercially available D-amino acid (available commercially in both protected and unprotected forms).

As used herein the terms “carrier” or “pharmaceutically acceptable carrier” refer to a diluent, adjuvant, excipient, or vehicle with which a compound/drug product/composition (including a formulation or medicament) is administered or formulated for administration. Non-limiting examples of such pharmaceutically acceptable carriers include liquids, such as water, saline, oils and solids, such as gum acacia, gelatin, starch paste, talc, keratin, colloidal silica, silica particles (nanoparticles or microparticles) urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

A capital letter “D” used in conjunction with an abbreviation for an amino acid residue refers to the D-form of the amino acid residue.

As used herein, the phrase “delaying the onset of” refers to, in a statistical sample, postponing, hindering the occurrence of a disease, disorder or condition, or causing one or more signs, symptoms or conditions of a disease, disorder or condition to occur more slowly than normal, in a sample or subject administered a therapeutic agent(s) relative to a control sample or subject.

The term “DMT” refers to 2′,6′-di(methyl) tyrosine (e.g., 2′,6′-dimethyl-L-tyrosine; CAS 123715-02-6).

As used herein, the term “effective amount” refers to a quantity of a compound/composition/drug product sufficient to achieve a desired therapeutic and/or prophylactic effect, e.g., an amount that treats, prevents, inhibits, ameliorates, or delays the onset of the disease, disorder or condition, or the physiological signs, symptoms or conditions of the disease or disorder. In the context of therapeutic or prophylactic applications, in some embodiments, the amount of a compound/composition/drug product administered to the subject will depend on the type and severity of the disease and on the characteristics of the individual, such as general health, age, sex, body weight and tolerance to drugs. In some embodiments, it will also depend on the degree, severity and type of disease. The skilled artisan will be able to determine appropriate dosages depending on these and other factors. The compounds/compositions/drug products can also be administered in combination with one or more additional therapeutic compounds/agents (a so called “co-administration” where, for example, the additional therapeutic agent(s) could be administered simultaneously, sequentially or by separate administration).

As used herein, the term “hydrate” refers to a compound which is associated (e.g., complexed) with water. The number of the water molecules contained in a hydrate of a compound may be (or may not be) in a definite ratio to the number of the compound molecules in the hydrate.

As used herein, “inhibit” or “inhibiting” refers to the reduction in a sign, symptom or condition (e.g. risk factor) associated with a disease or disorder associated with Friedreich's ataxia. In one embodiment, inhibit or inhibiting refers to the reduction by at least a statistically significant amount compared to a control (or control subject). In one embodiment, inhibit or inhibiting refers to a reduction by at least 5 percent compared to control (or control subject). In various individual embodiments, inhibit or inhibiting refers to a reduction by at least 1, 2, 3, 4, 5, 10, 15, 20, 25, 30, 33, 40, 50, 60, 67, 70, 75, 80, 90, 95, or 99 percent compared to a control (or control subject).

As used herein, the term “pharmaceutically acceptable salt” refers to a salt of a therapeutic compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]+). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic, p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66:1-19 (1977)). Certain specific compounds may contain both basic and acidic functionalities that allow the compounds to be converted into either base or acid addition salts or exist in zwitterionic form. These salts may be prepared by methods known to those skilled in the art. Other pharmaceutically acceptable carriers known to those of skill in the art are suitable for the present technology.

As used herein, “prevention” or “preventing” of a disease, disorder, or condition refers to results that, in a statistical sample, exhibit a reduction in the occurrence of the disease, disorder, or condition in a sample or subject administered a therapeutic agent relative to a control sample or subject. Such prevention is sometimes referred to as a prophylactic treatment.

As used herein, the term “separate” therapeutic use refers to an administration of at least two active ingredients (e.g. therapeutic agents) at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this definition.

As used herein, the term “simultaneous” therapeutic use refers to the administration of at least two active ingredients by the same route and at the same time or at substantially the same time.

As used herein, the term “solvate” refers to forms of the compound that are associated with a solvent, possibly by a solvolysis reaction. This physical association may include hydrogen bonding. Conventional solvents include water, methanol, ethanol, isopropanol, acetic acid, ethyl acetate, acetone, hexane(s), DMSO, THF, diethyl ether, and the like.

As used herein, a “subject” refers to a living animal. In various embodiments, a subject is a mammal. In various embodiments, a subject is a non-human mammal, including, without limitation, a mouse, rat, hamster, guinea pig, rabbit, sheep, goat, cat, dog, pig, minipig, horse, cow, or non-human primate. In certain embodiments, the subject is a human.

It is also to be appreciated that the various modes of treatment or prevention of medical conditions as described herein, in some embodiments, are intended to mean “substantial,” which includes total but also less than total treatment or prevention, and wherein some biologically or medically relevant result is achieved.

As used herein, a “synergistic therapeutic effect” refers to a greater-than-additive therapeutic effect which is produced by a combination of at least two agents, and which exceeds that which would otherwise result from the individual administration of the agents.

As used herein, the term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

As used herein, the terms “treating” or “treatment” refer to therapeutic treatment, wherein the object is to reduce, alleviate or slow down (lessen) a pre-existing disease or disorder, or its related signs, symptoms or conditions. By way of example, but not by way of limitation, a subject is successfully “treated” for a disease if, after receiving an effective amount of the compound/composition/drug product or a pharmaceutically acceptable salt, stereoisomer, tautomer, hydrate, and/or solvate thereof, the subject shows observable and/or measurable reduction in or absence of one or more signs, symptoms or conditions associated with the disease, disorder or condition. It is also to be appreciated that the various modes of treatment of medical conditions as described are intended to mean “substantial,” which includes total alleviation of conditions, signs or symptoms of the disease or disorder, as well as “partial,” where some biologically or medically relevant result is achieved.

### II. Pharmaceutical Compositions, Routes of Administration, and Dosing

The combination of elamipretide and omaveloxolone disclosed herein can be used, alone or in combination, with other therapeutic agent(s) to address the needs of subjects suffering from Friedreich's ataxia. In order to be administered to a subject in need thereof, the combination of elamipretide and omaveloxolone will generally need to be formulated (for individual administration or in a combined formulation) for the route of administration. In some embodiments, the same route of administration can be used to deliver both the omaveloxolone. In some embodiments, each of the elamipretide and omaveloxolone can be administered by different routes. The formulated product(s) can be considered a composition or medicament comprising the elamipretide and/or omaveloxolone (either formulated individually for separate administration or combined in a single formulation for simultaneous administration) and optionally one or more other (i.e., additional) therapeutic agents.

In some embodiments, the therapeutic agent(s) can be formulated with little or no excipient or carrier. In some embodiments, the therapeutic agent(s) can be formulated such that the majority of the formulation is excipient or carrier. In brief, one of skill in the art will tailor the formulation to have a suitable amount of excipient or carrier based on the needs/condition of the subject, the kind and extent of the disease to be treated; the properties of the therapeutic agent or agents to be delivered and the selected mode of administration of the particular therapeutic agent or agents.

In certain embodiments, a pharmaceutical composition may further comprise at least one therapeutic agent other than the elamipretide and/or omaveloxolone (e.g. an other (or additional) therapeutic agent for use in combination with the elamipretide and omaveloxolone). The at least one other/additional therapeutic agent can be an agent useful in the treatment of Friedreich's ataxia or could, for example, be administered to ameliorate the side effect of the administration of elamipretide and/or omaveloxolone (e.g., to address an injection site reaction to the administration of elamipretide if given subcutaneously). Thus, in some embodiments, pharmaceutical compositions can be prepared, for example, by combining elamipretide and/or omaveloxolone with a pharmaceutically acceptable carrier and, optionally, one or more additional therapeutical agents or otherwise merely administering the other/additional therapeutic agent(s) in combination with the administration or elamipretide and/or omaveloxolone.

Pharmaceutical compositions may contain an effective amount of one or more of the therapeutic agent or agents as described herein and may optionally be disbursed (e.g. dissolved, suspended or otherwise) in a pharmaceutically acceptable carrier. The components of the pharmaceutical composition(s) may also be capable of being commingled with the compounds of the present application, and with each other, in a manner such that there is no interaction that would substantially impair the desired pharmaceutical efficiency.

As stated above, an “effective amount” refers to any amount of a particular therapeutic agent that is sufficient to achieve a desired biological effect. Combined with the teachings provided herein, by choosing among the various therapeutic compound(s) and weighing factors such as potency, relative bioavailability, patient body weight, severity of adverse side-effects and mode of administration, an effective prophylactic (i.e. preventative) or therapeutic treatment regimen can be planned which does not cause substantial unwanted toxicity and yet is effective to address the particular condition, disorder or disease of a particular subject in a therapeutic way. The effective amount of a therapeutic agent for any particular indication can vary depending on such factors as the disease, disorder or condition being treated, the particular compound or compounds being administered, the size of the subject, the age of the subject, the overall health of the subject and/or the severity of the disease, disorder or condition. The effective amount may be determined during pre-clinical trials and/or clinical trials by methods familiar to physicians and clinicians. One of ordinary skill in the art can empirically determine the effective amount of a particular therapeutic agent or agents without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein. A dose may be administered by oneself, by another or by way of a device (e.g. a pump).

For any therapeutic compound described herein the therapeutically effective amount can, for example, be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

Therapeutic compounds (alone or as formulated in a pharmaceutical composition/medicament) for use in therapy or prevention can be tested in suitable animal model systems. Suitable animal model systems include, but are not limited to, rats, mice, chicken, cows, monkeys, rabbits, pigs, minipigs and the like, prior to testing in human subjects. In vivo testing, of any of the animal model system known in the art can be used prior to administration to human subjects. In some embodiments, dosing can be tested directly in humans.

Dosage, toxicity and therapeutic efficacy of any therapeutic agents or compositions (e.g. formulations or medicaments), other/additional therapeutic agents, or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, in such cases it may be prudent to design a delivery system that targets such compounds to the site of affected tissue in order to minimize potential damage to uninfected cells and, thereby, reduce side effects.

An exemplary treatment regime can entail administration once per day, twice per day, thrice per day, once a week, or once a month. In therapeutic applications, a relatively high dosage at relatively short intervals is sometimes required until progression of the disease is delayed, reduced or terminated, or until the subject shows partial or complete amelioration of symptoms of disease. Thereafter, the patient can be administered a prophylactic regimen.

For use in therapy, an effective amount of the therapeutic compound (alone or as formulated) can be administered to a subject by any mode that delivers the compound to the desired surface. Administering a pharmaceutical composition may be accomplished by any means known to the skilled artisan. Routes of administration include but are not limited to oral, topical, intranasal, systemic, intravenous, subcutaneous, intraperitoneal, intradermal, intraocular, ophthalmical, intrathecal, intracerebroventricular, iontophoretical, transmucosal, intravitreal, or intramuscular administration. Administration includes self-administration, administration by another and administration by a device (e.g. a pump).

A therapeutic compound/agent disclosed herein can be delivered to the subject in a formulation or medicament (i.e. a pharmaceutical composition). Formulations and medicaments can be prepared by, for example, dissolving or suspending a therapeutic compound/agent disclosed herein (e.g., elamipretide and/or omaveloxolone) in water, a pharmaceutically acceptable carrier, salt, (e.g. NaCl or sodium phosphate), buffering agents, preservatives, compatible carriers, adjuvants, and optionally other therapeutically acceptable ingredients.

The pharmaceutical compositions (e.g. a formulation or medicament) can include a carrier (e.g., a pharmaceutically acceptable carrier), which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars (e.g. trehalose), polyalcohols such as mannitol, sorbitol, or sodium chloride in the composition. Prolonged absorption of the injectable compositions can be brought about by including in the composition an agent that delays absorption, for example, aluminum monostearate or gelatin.

Solutions or suspensions (e.g., a formulation or medicament) used for parenteral, intradermal, subcutaneous or intraocular application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided alone or in a kit containing all necessary equipment (e.g., vials of drug, vials of diluent, syringes and needles) for a treatment course (e.g., 1, 2, 3, 4, 5, 6, 7 days or more of treatment).

The therapeutic compounds/agents or pharmaceutical compositions, when it is desirable to deliver them systemically, may be formulated for parenteral administration by injection, e.g., by bolus injection or continuous infusion (for example by IV injection or via a pump to meter the administration over a defined time). Formulations for injection may be presented in unit dosage form, e.g., in ampoules or in multi-dose containers, with an added preservative. Pharmaceutical compositions may take such forms as suspensions, solutions or emulsions in oily or aqueous vehicles, and may contain formulatory agents such as suspending, stabilizing and/or dispersing agents. Additionally, suspensions of the therapeutic compounds (e.g., elamipretide and/or omaveloxolone) may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Systemic formulations include those designed for administration by injection, e.g., subcutaneous, intravenous, intramuscular, intrathecal or intraperitoneal injection, as well as those designed for transdermal, transmucosal oral or pulmonary administration.

For intravenous and other parenteral routes of administration, a compound (e.g., elamipretide and/or omaveloxolone) can be formulated as a lyophilized preparation, as a lyophilized preparation of liposome-intercalated or lipid-encapsulated therapeutic compound(s), as a lipid complex in aqueous suspension, or as a salt complex. Lyophilized formulations are generally reconstituted in suitable aqueous solution, e.g., in sterile water or saline, shortly prior to administration.

Pharmaceutical compositions (e.g., a formulation or medicament) suitable for injection can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). A composition for administration by injection will generally be sterile and should be fluid to the extent that easy syringability exists. It should be stable under the conditions of manufacture and storage and may be preserved against the contaminating action of microorganisms such as bacteria and fungi.

Sterile injectable solutions (e.g., a formulation or medicament) can be prepared by incorporating the therapeutic compound(s) (e.g., elamipretide and/or omaveloxolone) in the required amount in an appropriate solvent with one or a combination of ingredients enumerated above, as required, followed by filtered sterilization. Generally, dispersions are prepared by incorporating the therapeutic compound(s) into a sterile vehicle, that contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

For oral administration, the compounds can be formulated readily by combining the therapeutic compound(s) (e.g., elamipretide and/or omaveloxolone) with pharmaceutically acceptable carriers well known in the art. Such carriers enable the therapeutic compound(s) to be formulated as tablets, pills, dragees, capsules, liquids, gels, syrups, slurries, suspensions and the like, for oral ingestion by a subject to be treated. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or sterates; a glidant such as colloidal silicon dioxide; a sweetening agent such as sucrose or saccharin; or a flavoring agent such as peppermint, methyl salicylate, or orange flavoring.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above that may be chemically modified so that oral delivery of the derivative is efficacious. Generally, the chemical modification contemplated is the attachment of at least one moiety to the therapeutic agent(s), ingredient(s), and/or excipient(s), where said moiety permits (a) inhibition of acid hydrolysis; and (b) uptake into the blood stream from the stomach or intestine. Also desired is the increase in overall stability of the therapeutic agent(s), ingredient(s), and/or excipient(s) and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol (PEG) moieties of various molecular weights are suitable.

For the formulation of the therapeutic agent(s), ingredient(s), and/or excipient(s), the location of release may be the stomach, the small intestine (the duodenum, the jejunum, or the ileum), or the large intestine. One skilled in the art has available formulations which will not dissolve in the stomach yet will release the material in the duodenum or elsewhere in the intestine. Preferably, the release will avoid the deleterious effects of the stomach environment, either by protection of a therapeutic compound/agent or by release of the biologically active material beyond the stomach environment, such as in the intestine.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic compound(s)/agent(s) (which term “therapeutic compound(s)/agent(s)” as used herein is intended to refer to elamipretide and/or omaveloxolone and any other active pharmaceutical ingredient (e.g., other therapeutic agent) that can be administered in a combination with elamipretide and/or omaveloxolone) or pharmaceutical composition(s) can be included in the formulation as fine multi-particulates in the form of granules or pellets of particle size about 1-2 mm. The formulation of the material for capsule administration could also be as a powder, lightly compressed plugs or even as tablets. The therapeutic compound(s)/agent(s) or pharmaceutical composition(s) could be prepared by compression.

Colorants and flavoring agents may all be included. For example, the therapeutic compound(s)/agent(s) or pharmaceutical composition(s) may be formulated and then further contained within an edible product, such as a refrigerated beverage containing colorants and flavoring agents.

One may dilute or increase the volume of the therapeutic compound(s)/agent(s) or pharmaceutical composition(s) with an inert material. These diluents could include carbohydrates, especially mannitol, lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

Disintegrants may be included in the formulation of the therapeutic compound(s)/agent(s) or pharmaceutical composition(s) into a solid dosage form. Materials used as disintegrates include but are not limited to starch, including the commercial disintegrant based on starch, Explotab. Sodium starch glycolate, Amberlite®, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, karaya gum or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic agent(s).

An anti-frictional agent may be included in the formulation of the therapeutic compound(s)/agent(s) or pharmaceutical composition(s) to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol (PEG) of various molecular weights, Carbowax™ 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic compound(s)/agent(s) or pharmaceutical composition(s) into the aqueous environment, a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation or medicament as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation or medicament disclosed herein or derivative either alone or as a mixture in different ratios.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the therapeutic compound(s) may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the therapeutic compound(s)/agent(s) or pharmaceutical composition(s) may be formulated as solutions, gels, ointments, creams, suspensions, etc. as are well-known in the art. Solutions, gels, ointments, creams or suspensions may be administered topically. The compounds may also be formulated in rectal or vaginal compositions such as suppositories or retention enemas, e.g., containing conventional suppository bases such as cocoa butter or other glycerides.

For administration by inhalation, therapeutic compound(s)/agent(s) or pharmaceutical composition(s) for use according to the present application may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the formulation, medicament and/or therapeutic compound(s)/agent(s) can be delivered in the form of an aerosol spray from a pressurized container or dispenser, which contains a suitable propellant, e.g., a gas such as carbon dioxide, or a nebulizer. Such methods include those described in U.S. Pat. No. 6,468,798. In the case of a pressurized aerosol the dosage unit may be determined by providing a valve to deliver a metered amount. In the case of a pressurized aerosol the dosage unit may be determined by providing a valve to deliver a metered amount. For example, capsules and cartridges of e.g., gelatin for use in an inhaler or insufflator may be formulated containing a powder mix of the therapeutic compound/agent and a suitable powder base such as lactose or starch. Alternatively, the therapeutic compound(s)/agent(s) or pharmaceutical composition(s) may be in powder form for constitution with a suitable vehicle, e.g., sterile pyrogen-free water, before use.

Nasal delivery of a therapeutic compound(s)/agent(s) or pharmaceutical composition(s) is also contemplated. Nasal delivery allows the passage of therapeutic compound(s)/agent(s) or pharmaceutical composition(s) to the blood stream directly after administering the therapeutic compound(s)/agent(s) or pharmaceutical composition(s) to the nose, without the necessity for deposition of the product in the lung. Formulations for nasal delivery include those with dextran or cyclodextran.

For nasal administration, one type of useful device is a small, hard bottle to which a metered dose sprayer is attached. In some embodiments, the metered dose is delivered by drawing a pharmaceutical composition (in solution form) into a chamber of defined volume, which chamber has an aperture dimensioned to aerosolize and aerosol formulation by forming a spray when a liquid in the chamber is compressed. The chamber is compressed to administer the therapeutic compound(s)/agent(s) or pharmaceutical composition(s). In a specific embodiment, the chamber is a piston arrangement. Such devices are commercially available.

Alternatively, a plastic squeeze bottle with an aperture or opening dimensioned to aerosolize an aerosol formulation by forming a spray when squeezed can be used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the therapeutic compound(s)/agent(s) or pharmaceutical composition(s).

Also contemplated herein is pulmonary delivery of the compounds disclosed herein. The therapeutic compound(s)/agent(s) or pharmaceutical composition(s) is/are delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5): 143-146 (1989) (endothelin-1); Hubbard et al., Annal Int Med 3:206-212 (1989) (a1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995, to Wong et al.

Contemplated for use in the practice of this technology are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this technology are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

All such devices require the use of formulations suitable for the dispensing of the therapeutic compound(s)/agent(s) or pharmaceutical composition(s). Typically, each formulation is specific to the type of device employed and may involve the use of an appropriate propellant material, in addition to the usual diluents, adjuvants and/or carriers useful in therapy. Also, the use of liposomes, microcapsules, microspheres, nanoparticles, nanospheres, inclusion complexes, or other types of carriers is contemplated.

Formulations suitable for use with a nebulizer, either jet or ultrasonic, can, for example, comprise therapeutic compound(s)/agent(s) or pharmaceutical composition(s) dissolved in water at a concentration of about 0.01 to 50 mg of biologically active compound per mL of solution. The formulation may also include a buffer and optionally a simple sugar (e.g., for inhibitor stabilization and regulation of osmotic pressure). The nebulizer formulation may also contain a surfactant, to reduce or prevent surface induced aggregation of the therapeutic compound(s)/agent(s) or pharmaceutical composition(s) disclosed herein caused by atomization of the solution in forming the aerosol.

Formulations for use with a metered-dose inhaler device may generally comprise a finely divided powder comprising the therapeutic compound(s)/agent(s) or pharmaceutical composition(s) disclosed herein suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device may comprise a finely divided dry powder containing the therapeutic compound(s)/agent(s) or pharmaceutical composition(s) and may also include a bulking agent, such as lactose, sorbitol, sucrose, or mannitol in amounts which facilitate dispersal of the powder from the device, e.g., 50 to 90% by weight of the formulation. The compound(s)/therapeutic agent(s)/pharmaceutical composition(s) can advantageously be prepared in particulate or nanoparticulate form with an average particle size of less than 10 micrometers (μm), most preferably 0.5 to 5 μm, for most effective delivery to the deep lung.

For ophthalmic or intraocular indications, any suitable mode of delivering the therapeutic compound(s)/agent(s) or pharmaceutical composition(s) to the eye or regions near the eye can be used. For ophthalmic formulations generally, see Mitra (ed.), Ophthalmic Drug Delivery Systems, Marcel Dekker, Inc., New York, N.Y. (1993) and also Havener, W. H., Ocular Pharmacology, C. V. Mosby Co., St. Louis (1983). Nonlimiting examples of pharmaceutical compositions suitable for administration in or near the eye include, but are not limited to, ocular inserts, minitablets, and topical formulations such as eye drops, ointments, and in situ gels. In one embodiment, a contact lens is coated with a pharmaceutical composition (or contains a pharmaceutical composition encapsulated therein) comprising a therapeutic compound/agent. In some embodiments, a single dose can comprise from between 0.1 ng to 5000 μg, 1 ng to 500 μg, or 10 ng to 100 μg of the therapeutic compound(s)/agent(s) or pharmaceutical composition(s) administered to the eye.

Eye drops can comprise a sterile liquid formulation that can be administered directly to the eye. In some embodiments, eye drops comprise at least one therapeutic agent and may further comprise one or more preservatives. In some embodiments, the optimum pH for eye drops equals that of tear fluid and is about 7.4. For eye drops, the therapeutic compound(s)/agent(s) can be present in the drop solution from about 0.1% to about 5% (w/v or v/v depending on the physical nature (i.e. solid or liquid) of the active ingredient). In some embodiments, the therapeutic compound/agent can be present in the drop solution from about 1% to about 3% (w/v or v/v, as appropriate).

In situ gels are viscous liquids, showing the ability to undergo sol-to-gel transitions when influenced by external factors, such as appropriate pH, temperature, pressure and/or the presence of electrolytes. This property causes slowing of drug drainage from the eyeball surface and increase of the active ingredient bioavailability. Polymers commonly used in in situ gel formulations include, but are not limited to, gellan gum, poloxamer, silicone containing formulations, silica-based formulations and cellulose acetate phthalate. In some embodiments, the therapeutic compound(s)/agent(s) or pharmaceutical composition(s) is/are formulated into an in-situ gel (as the formulation/medicament).

For topical ophthalmic administration, therapeutic compound(s)/agent(s) or pharmaceutical composition(s) is/are may be formulated as solutions, gels, ointments, creams, suspensions, etc. as are well-known in the art. Ointments are semisolid dosage forms for external use such as topical use for the eye or skin. In some embodiments, ointments comprise a solid or semisolid hydrocarbon base of melting or softening point close to human core temperature. In some embodiments, an ointment applied to the eye decomposes into small drops, which stay for a longer time period in conjunctival sac, thus increasing bioavailability.

Ocular inserts are solid or semisolid dosage forms without disadvantages of traditional ophthalmic drug forms. They are less susceptible to defense mechanisms like outflow through nasolacrimal duct, show the ability to stay in conjunctival sac for a longer period, and can be more stable than conventional dosage forms. They also offer advantages such as accurate dosing of one or more therapeutic compound(s)/agent(s) or pharmaceutical composition(s), slow release of one or more therapeutic compound(s)/agent(s) with constant speed and limiting of one or more therapeutic compounds'/agents' systemic absorption. In some embodiments, an ocular insert comprises one or more therapeutic compound(s)/agent(s) and one or more polymeric materials. The polymeric materials can include, but are not limited to, methylcellulose and its derivatives (e.g., hydroxypropyl methylcellulose (HPMC)), ethylcellulose, polyvinylpyrrolidone (PVP K-90), polyvinyl alcohol, chitosan, carboxymethyl chitosan, gelatin, and various mixtures of the aforementioned polymers. An ocular insert can comprise silica. An ocular insert can comprise liposomes, nanoparticles or microparticles of degradable or biodegradable polymer (as described in more detail below).

Minitablets are biodegradable, solid drug forms, that transit into gels after application to the conjunctival sac, thereby extending the period of contact between active ingredient (i.e., the therapeutic compound(s)/agent(s)) and the eyeball surface, which in turn increases a therapeutic compounds'/agents' bioavailability. The advantages of minitablets include easy application to conjunctival sac, resistance to defense mechanisms like tearing or outflow through nasolacrimal duct, longer contact with the cornea caused by presence of mucoadhesive polymers, and gradual release of the active ingredient from the formulation in the place of application due to the swelling of the outer carrier layers. Minitablets can comprise one or more therapeutic compound(s)/agent(s) and one or more polymers. Nonlimiting examples of polymers suitable for use in in a minitablet formulation include cellulose derivatives, like hydroxypropyl methylcellulose (HPMC), hydroxyethyl cellulose (HEC), sodium carboxymethyl cellulose, ethyl cellulose, acrylates (e.g., polyacrylic acid and its cross-linked forms), Carbopol® or carbomer, chitosan, and starch (e.g., drum-dried waxy maize starch). In some embodiments, minitablets further comprise one or more excipients. Nonlimiting examples of excipients include mannitol and magnesium stearate.

The ophthalmic or intraocular formulations and medicaments may contain non-toxic auxiliary substances such as antibacterial components which are generally non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid (EDTA), and the like.

In some embodiments, the viscosity of the ocular formulation comprising one or more therapeutic compound(s)/agent(s) is increased to improve contact with the cornea and bioavailability in the eye. Viscosity can be increased by the addition of hydrophilic polymers of high molecular weight which do not diffuse through biological membranes and which form three-dimensional networks in the water. Nonlimiting examples of such polymers include polyvinyl alcohol, poloxamers, hyaluronic acid, carbomers, and polysaccharides, cellulose derivatives, gellan gum, and xanthan gum.

In addition to the formulations described above, therapeutic compound(s)/agent(s) or pharmaceutical composition(s) may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

In some embodiments, the therapeutic compound(s)/agent(s) or pharmaceutical composition(s) is/are administered as a depot formulation wherein the active therapeutic agent(s) is/are encapsulated by, or disposed within, silica-based microparticles. Such a formulation may be a controlled-release, delayed-release or extended release formulation (terms are defined below). Such controlled-release, delayed release or extended release formulation may comprise particles, such as microparticles or nanoparticles.

The pharmaceutical compositions also may comprise suitable solid or gel-phase carriers or excipients. Examples of such carriers or excipients include but are not limited to calcium carbonate, calcium phosphate, various sugars, starches, cellulose derivatives, gelatin, silica/silicone and polymers such as polyethylene glycols.

Suitable liquid or solid pharmaceutical preparation forms (e.g., a formulation or medicament) can, for example, be aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions/formulations may also include granules, powders, tablets, coated tablets, (micro) capsules, suppositories, syrups, emulsions, suspensions, creams, drops or preparations with protracted release of therapeutic compound(s), in whose preparation excipients and additives and/or auxiliaries such as disintegrants, binders, coating agents, swelling agents, lubricants, flavorings, sweeteners or solubilizers are customarily used as described above. The pharmaceutical compositions can be suitable for use in a variety of drug delivery systems. For a brief review of methods for drug delivery, see Langer R, Science 249:1527-33 (1990).

The therapeutic compound(s)/agent(s) or pharmaceutical composition(s) may be provided in particles. Particles as used herein means nanoparticles or microparticles (or in some instances larger particles) which can consist in whole or in part of the therapeutic compound(s)/agent(s) as described herein. The particles may contain the therapeutic compound(s)/agent(s) in a core surrounded by a coating, including, but not limited to, an enteric coating. The therapeutic compound(s)/agent(s) also may be dispersed throughout the particles. The therapeutic compound(s)/agent(s) also may be adsorbed into the particles. The particles may be of any order release kinetics, including zero-order release, first-order release, second-order release, delayed release, sustained release, immediate release, and any combination thereof, etc. The particle may include, in addition to any therapeutic compound(s)/agent(s), any of those materials routinely used in the art of pharmacy and medicine, including, but not limited to, erodible, non-erodible, biodegradable, or nonbiodegradable material or combinations thereof. The particles may be microcapsules which contain the therapeutic compound(s)/agent(s) in a solution or in a semi-solid state. The particles may be of virtually any shape.

Both non-biodegradable and biodegradable polymeric materials can be used in the manufacture of particles for delivering the therapeutic compound(s)/agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, polyethylene glycols (PEGs), polyvinylalcohols (PVAs), poly(methyl methacrylates), poly(ethyl methacrylates), poly(butylmethacrylate), poly(isobutyl methacrylate), poly-lactic acid (PLA), poly(lactic-co-glycolic) acid (PLGA), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) and poly(ε-caprolactone) or mixtures of two or more of the foregoing. The biodegradable polymeric materials may be substantially pure single polymer or mixes of two or more polymers wherein the materials comprise mixtures of single monomers, block co-polymers or a mixture thereof.

Therapeutic compound(s)/agent(s) or mixtures of two or more therapeutic compound(s)/agent(s) can be formulated in a carrier system. The carrier can be a colloidal system. The carrier or colloidal system can be a liposome, a phospholipid bilayer vehicle. In one embodiment, therapeutic compound(s)/agent(s) or mixtures of two or more therapeutic compound(s)/agent(s) can be encapsulated in a liposome while maintaining integrity of the therapeutic compound(s)/agent(s). One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34 (7-8): 915-923 (2000)). For example, a therapeutic agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic compound(s)/agent(s) or mixtures of two or more therapeutic compound(s)/agent(s) can be embedded in the polymer matrix, while maintaining integrity of the composition. The polymer can be a microparticle or nanoparticle that encapsulates therapeutic compound(s)/agent(s). The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In some embodiments, the polymer is poly-lactic acid (PLA), poly lactic/glycolic acid (PLGA) or a mixture thereof. The polymeric matrices can be prepared and isolated in a variety of forms and sizes, including microspheres and nanospheres. Polymer formulations can lead to prolonged duration of therapeutic effect. (See Reddy, Ann. Pharmacother., 34 (7-8): 915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the nanoparticles or microparticles can be silica-based or silane-based (See for example: WO2002/080977 entitled: “Biodegradable carrier and method for preparation thereof”).

In some embodiments, the therapeutic compound(s)/agent(s) or mixtures of two or more therapeutic compound(s)/agent(s) can be prepared with carriers that will protect the therapeutic compound(s)/agent(s) or other therapeutic agent(s) or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compound(s)/agent(s) or mixtures of two or more therapeutic compound(s)/agent(s) may be contained in controlled release systems. The term “controlled-release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained-release” (also referred to as “extended-release”) is used in its conventional sense to refer to a drug formulation that provides for gradual release of a drug over an extended period of time, and that preferably, although not necessarily, results in substantially constant blood levels of a drug over an extended time period. The term “delayed-release” is used in its conventional sense to refer to a drug formulation in which there is a time delay between administration of the formulation and the release of the drug therefrom to thereby make it available to the subject. “Delayed release” may or may not involve gradual release of drug over an extended period of time, and thus may or may not be “sustained release.”

Use of a long-term controlled-release or sustained-release implant or depot formulation may be particularly suitable for treatment of chronic conditions. The term “implant” and “depot formulation” is intended to include a single composition (such as a mesh) or composition comprising multiple components (e.g., a fibrous mesh constructed from several individual pieces of mesh material) or a plurality of individual compositions where the plurality remains localized and provides the long-term sustained-release of active pharmaceutical ingredient(s) occurring from the aggregate of the one or plurality of compositions. “Long-term” release, as used herein, means that the implant or depot formulation is constructed and arranged to deliver therapeutic or prophylactic levels of the active pharmaceutical ingredient(s) for at least 2 days. In some embodiments, the implant or depot formulation is constructed and arranged to deliver therapeutic or prophylactic levels of the active pharmaceutical ingredient(s) for at least 7 days. In some embodiments, the implant or depot formulation is constructed and arranged to deliver therapeutic or prophylactic levels of the active pharmaceutical ingredient(s) for at least 14 days. In some embodiments, the implant or depot formulation is constructed and arranged to deliver therapeutic or prophylactic levels of the active pharmaceutical ingredient(s) for at least 30 days. In some embodiments, the implant or depot formulation is constructed and arranged to deliver therapeutic or prophylactic levels of the active pharmaceutical ingredient(s) for at least 60 days. In some embodiments, the implant or depot formulation is constructed and arranged to deliver therapeutic or prophylactic levels of the active pharmaceutical ingredient for at least 90 days. In some embodiments, the implant or depot formulation is constructed and arranged to deliver therapeutic or prophylactic levels of the active pharmaceutical ingredient(s) for at least 180 days. In some embodiments, the implant or depot formulation is constructed and arranged to deliver therapeutic or prophylactic levels of the active pharmaceutical ingredient(s) for at least one year. In some embodiments, the implant or depot formulation is constructed and arranged to deliver therapeutic or prophylactic levels of the active pharmaceutical ingredient(s) for 15-30 days. In some embodiments, the implant or depot formulation is constructed and arranged to deliver therapeutic or prophylactic levels of the active pharmaceutical ingredient(s) for 30-60 days. In some embodiments, the implant or depot formulation is constructed and arranged to deliver therapeutic or prophylactic levels of the active pharmaceutical ingredient(s) for 60-90 days. In some embodiments, the implant or depot formulation is constructed and arranged to deliver therapeutic or prophylactic levels of the active pharmaceutical ingredient(s) for 90-120 days. In some embodiments, the implant or depot formulation is constructed and arranged to deliver therapeutic or prophylactic levels of the active pharmaceutical ingredient(s) for 120-180 days. In some embodiments, the implant or depot formulation is constructed and arranged to deliver therapeutic or prophylactic levels of the active pharmaceutical ingredient(s) for up to one year. In some embodiments, the long-term sustained-release implants or depot formulation are well-known to those of ordinary skill in the art and include some of the release systems described above. In some embodiments, such implants or depot formulation can be administered surgically. In some embodiments, such implants or depot formulation can be administered topically or by injection.

### III. Formulations and Medicaments

The combination of elamipretide and omaveloxolone disclosed herein can be used, alone or in combination, with one or more other therapeutic agents to address the needs of subjects suffering from Friedreich's ataxia. In order to be administered to a subject in need thereof, the elamipretide, omaveloxolone and/or other therapeutic agent(s) will generally need to be formulated for the suitable route of administration. For example, if the elamipretide, omaveloxolone and/or other therapeutic agent(s) is/are to be administered to the subject by injection, it/they will typically be formulated into an injectable liquid or liquid suspension. For example, this could be accomplished by dissolving or suspending the therapeutic agent(s) in a suitable diluent, adjuvant, excipient, vehicle or pharmaceutically acceptable carrier as described previously herein (See the section above entitled: Pharmaceutical Compositions, Routes of Administration, and Dosing). In some embodiments, the diluent, adjuvant, excipient, vehicle or pharmaceutically acceptable carrier can be water, saline or a buffered aqueous solution. Suitable methods, reagents and compositions for formulating the elamipretide, omaveloxolone and/or other therapeutic agent(s) into a suitable medicament are discussed above.

Similarly, if the elamipretide, omaveloxolone and/or other therapeutic agent(s) can be to be administered to the subject in oral form, the selected active ingredient(s) can be formulated into a pill, tablet, capsule or other vehicle for such administration as discussed above in the section entitled: “Pharmaceutical Compositions, Routes of Administration, and Dosing” or as otherwise known to those of ordinary skill in the art. Suitable methods, reagents and compositions for formulating the elamipretide, omaveloxolone and/or other therapeutic agent(s) into a suitable orally administrable medicament are discussed above.

Similarly, the elamipretide, omaveloxolone and/or other therapeutic agent(s) can be formulated for ocular administration, buccal administration, topical administration, nasal administration or any other of the modes of administration previously discussed herein or that are known to those of ordinary skill in the art. Suitable methods, reagents and compositions for formulating the elamipretide, omaveloxolone and/or other therapeutic agent(s) into a suitable ocular, buccal, topical, or nasal administrable medicament are discussed above.

In brief, any of the formulations (which can also be referred to as a medicament or composition when formulated for administration to a subject having a certain affliction or medical condition that requires medical attention) described in the section above entitled: “Pharmaceutical Compositions, Routes of Administration, and Dosing” can be applied to produce a composition (i.e. a formulation or medicament) suitable for administration to a subject in need thereof. Thus, in some embodiments, this application is directed to compositions, formulations and medicaments suitable for administration to a subject suffering from, or believed to be suffering from, Friedreich's ataxia.

In some embodiments, the formulation or medicament is administered subcutaneously. In some embodiments, the formulation or medicament is administered orally, topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, the elamipretide, omaveloxolone and/or other therapeutic agent(s) can be administered in a pharmaceutically acceptable salt form. In some embodiments, the pharmaceutically acceptable salt form comprises a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt or a maleate salt (in each case a mono-, bis- or tri-(tris-) acid salt). In some embodiments, pharmaceutically acceptable salt comprises a monoacetate salt, a bis-acetate salt, a tri-acetate salt, a mono-trifluoroacetate salt, a bis-trifluoroacetate salt, a tri-trifluoroacetate salt, a monohydrochloride salt, a bis-hydrochloride salt, a trihydrochloride salt, a mono-tosylate salt, a bis-tosylate salt, or a tri-tosylate salt. In some embodiments, elamipretide, omaveloxolone and/or other therapeutic agent(s) is/are formulated as a tris-HCl salt, a bis-HCl salt, or a mono-HCl salt.

### IV. Therapeutic Methods And Related Combination Product

In one aspect, the present disclosure provides a method for treating, preventing, inhibiting, ameliorating or delaying the onset of Friedreich's ataxia or the signs or symptoms of reduced frataxin levels or activity in a subject in need thereof, comprising administering to the subject a therapeutically effective amount of both elamipretide and omaveloxolone, or pharmaceutically acceptable salts, stereoisomers, mixtures of stereoisomers, tautomers, hydrates, and/or solvates thereof. In some embodiments of the method, the subject displays reduced levels of frataxin expression compared to a normal control subject. In some embodiments of the method, the Friedreich's ataxia comprises one or more of muscle weakness, loss of coordination, vision impairment, hearing impairment, slurred speech, curvature of the spine, diabetes, and heart disorders. In some embodiments of the method, the subject has been diagnosed as having Friedreich's ataxia. In some embodiments of the method, the subject is human.

In some embodiments of the method, administration of elamipretide and omaveloxolone to a subject diagnosed as having, or suspected of having, Friedreich's ataxia is effective to increase intracellular adenosine triphosphate (ATP) levels in tissue in a subject. In some embodiments of the method, administration of elamipretide and omaveloxolone to a subject diagnosed as having, or suspected of having, Friedreich's ataxia is effective to increase or maintain frataxin levels in the subject.

In some embodiments of the method, each of the elamipretide and omaveloxolone is independently administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly. In some embodiments of the method, the elamipretide and omaveloxolone are administered simultaneously, or substantially simultaneously. In some embodiments of the method, the elamipretide and omaveloxolone are administered sequentially in either order. In some embodiments of the method, the elamipretide and omaveloxolone are administered separately, simultaneously or sequentially in either order. In some embodiments of the method, the elamipretide and omaveloxolone are administered by different routes of administration. In some embodiments of the method, the elamipretide is administered subcutaneously. In some embodiments of the method, the omaveloxolone is administered orally. In some embodiments of the method, the administration of both elamipretide and omaveloxolone produces a synergistic therapeutic effect in the subject.

In another aspect, present disclosure provides a kit comprising both elamipretide and omaveloxolone, each in a form suitable for administration to a subject diagnosed as having, or suspected of having, Friedreich's ataxia. The kit can comprise a single dose of both elamipretide and omaveloxolone, or the kit can provide multiple doses of both elamipretide and omaveloxolone. The kit can comprise a single formulation or medicament containing both elamipretide and omaveloxolone or a formulation/medicament for elamipretide that is different from the formulation/medicament for omaveloxolone.

### EXAMPLES

The present technology is further illustrated by the following examples, which should not be construed as limiting in any way.

### Example 1: Cytoprotection Assay

### Introduction:

The purpose of this assay was to assess whether or not healthy or FA fibroblasts subjected to an insult with RLS3 (a compound known to induce ferroptosis) would exhibit a treatment effect upon treatment with elamipretide alone, omaveloxolone alone or the combination of omaveloxolone and elamipretide.

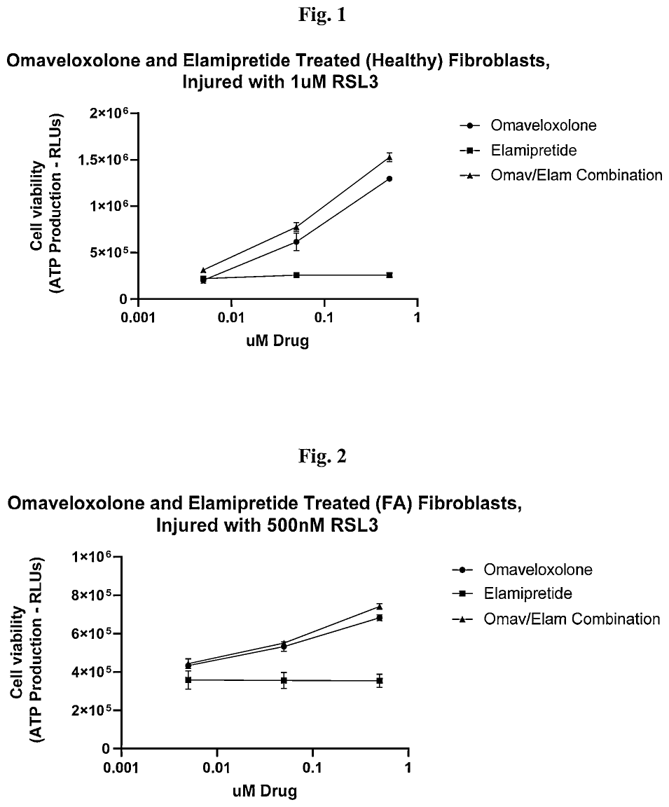

To initiate the cytoprotection experiment, healthy (GM08402; Coriell) and FA (GM03665; Coriell) fibroblasts were resuspended in assay medium (Minimum Essential Medium Eagle w/Earles Salts and L-glutamine (MEM; Corning)+15% Qualified Fetal Bovine Serum (FBS)+penicillin (100 U)/streptomycin (100 g), seeded at 10,000 cells per well in a clear, tissue cultured treated 96 well plate), and incubated at 37° C. Eight hours later, the assay medium was manually removed and test compounds, elamipretide (Stealth Biotherapeutics Inc., Needham, MA) and omaveloxolone were added to a final volume of 100 uL at concentrations ranging between 0.001 uM and 1 uM. Following an 18 hour incubation at 37° C., the assay medium was again manually removed. Healthy fibroblasts and FA fibroblasts were then treated with 100 μL of 1 uM and 0.5 uM RSL3 (Sigma, Catalog #1219810), respectively, in assay medium for 4 hours at 37° C. Cell viability was assessed with CellTiter-Glo Luminescent Cell Viability (Promega). Luminescence units were quantified by GEN5 software on a BioTek SYNERGY neo2 plate reader (LUM filter). The data obtained for the health cells are graphically illustrated in FIG. 1 and the data obtained for the FA fibroblasts are graphically illustrated in FIG. 2.

Note: The FA fibroblasts were treated with a lower concentration of RSL3 than the healthy patient cells because they are more susceptible to injury by the reagent.

### Results:

With reference to FIG. 1, in the healthy patient derived fibroblasts, treatment with elamipretide alone exhibited no apparent effect on the RSL3 treated cells. In contrast, treatment with omaveloxolone treatment appeared to provide a significant protective/rescue effect.

Additionally, the combination of elamipretide and omaveloxolone appeared to provide even greater protective/rescue effect. As elamipretide alone had no apparent affect at all, the combination of elamipretide and omaveloxolone appears to provide better than just an additive effect and therefore the combination of omaveloxolone and elamipretide appears to produce a synergistic therapeutic effect in the healthy patient cell line.

Similarly with reference to FIG. 2, in the FA patient derived fibroblasts, treatment with elamipretide alone exhibited no apparent effect on the RSL3 treated cells. In contrast, treatment with omaveloxolone treatment appeared to provide a significant protective/rescue effect. Additionally, the combination of elamipretide and omaveloxolone appeared to provide even greater protective/rescue effect, albeit only slightly improved over omaveloxolone, alone. As elamipretide alone had no apparent affect at all, the combination of elamipretide and omaveloxolone appears to provide better than just an additive effect and therefore the combination of omaveloxolone and elamipretide appears to produce a synergistic therapeutic effect in the FA fibroblasts.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a nonlimiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

1. A method for treating, preventing, inhibiting, ameliorating or delaying the onset of Friedreich's ataxia or the signs or symptoms of reduced frataxin levels or activity in a subject in need thereof, comprising administering to the subject a therapeutically effective amount of both elamipretide and omaveloxolone, or pharmaceutically acceptable salts, stereoisomers, mixtures of stereoisomers, tautomers, hydrates, and/or solvates thereof.

2. The method of claim 1, wherein the subject displays reduced levels of frataxin expression compared to a normal control subject.

3. The method of claim 1, wherein the Friedreich's ataxia comprises one or more of muscle weakness, loss of coordination, vision impairment, hearing impairment, slurred speech, curvature of the spine, diabetes, and heart disorders.

4. The method of claim 1, wherein administration of elamipretide and omaveloxolone to a subject diagnosed as having, or suspected of having, Friedreich's ataxia is effective to increase intracellular adenosine triphosphate (ATP) levels in tissue in a subject.

5. The method of claim 1, wherein administration of elamipretide and omaveloxolone to a subject diagnosed as having, or suspected of having, Friedreich's ataxia is effective to increase or maintain frataxin levels in the subject.

6. The method of claim 1, wherein each of the elamipretide and omaveloxolone is independently administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly.

7. The method of claim 1, wherein the elamipretide and omaveloxolone are administered simultaneously, or substantially simultaneously.

8. The method of claim 1, wherein the elamipretide and omaveloxolone are administered sequentially in either order.

9. The method of claim 1, wherein the elamipretide and omaveloxolone are administered separately.

10. The method of claim 1, wherein the elamipretide and omaveloxolone are administered by different routes of administration.

11. The method of claim 10, wherein the elamipretide is administered subcutaneously.

12. The method of claim 10, wherein the omaveloxolone is administered orally.

13. The method of claim 1, wherein the subject has been diagnosed as having Friedreich's ataxia.

14. The method of claim 1, wherein the subject is human.

15. The method of claim 1, wherein the administration of both elamipretide and omaveloxolone produces a synergistic therapeutic effect.

16. A kit comprising both elamipretide and omaveloxolone, or pharmaceutically acceptable salts, stereoisomers, mixtures of stereoisomers, tautomers, hydrates, and/or solvates thereof, each in a form suitable for administration to a subject diagnosed as having, or suspected of having, Friedreich's ataxia.

17. A composition, formulation or medicament comprising both elamipretide and omaveloxolone, or pharmaceutically acceptable salts, stereoisomers, mixtures of stereoisomers, tautomers, hydrates, and/or solvates thereof, in a form suitable for administration to a subject diagnosed as having, or suspected of having, Friedreich's ataxia.
